(19) United States
(12) Patent Application Publication
(10) Pub. No.: US 2010/0198081 A1
(43) Pub. Date: Aug. 5, 2010

(54) Scanning light imager

(76) Inventors: John Harold Hanlin, Louisville, CO (US); David Charles Amundson, Louisville, CO (US)

Correspondence Address: David Amundson, 192 Regal St, Louisville, CO, 80027, US

(21) Appl. No.: 12/322,317

(22) Filed: Feb. 2, 2009

(51) Int. Cl. A61B 6/00

(52) U.S. Cl. 600/478

(57) ABSTRACT

This invention describes the detection of atherosclerotic plaque or cancer cells by a light probe inside a blood vessel or internal to an elongate organ. In one embodiment, vessel wall is imaged by employing a scanning mechanism using one emitting and one receiving fiber, whereby light is directed at a spinning mirror, approximately normal to the vessel or elongate organ surface. The light is reflected circumferentially around the vessel or elongate organ surface as the mirror rotates and received by a low-numerical aperture (NA) fiber, which transmits it to a light detector, thereby generating a set of light amplitudes circumferentially around the vessel/elongate organ surface. Multiple rings are acquired by translating the probe within the vessel/elongate organ. In another embodiment, adding a piezoelectric transducer in proximity to the distal ends of the fibers permits simultaneous ultrasound and light images to be created.

### FIELD OF INVENTION

This invention relates to a scanning technique of light imaging of vascular wall obscured by blood and in detecting feint objects, such as cancer internally in elongate organs.

### REFERENCES

Haney, D. Vulnerable plaque: the latest in heart disease. Assoc Press; Jan. 11, 1999 Hatsukami, T S, Ross, R, Nayak, P L, Yuan, C. Visualization of fibrous cap thickness and rupture in human atherosclerotic carotid plaque in vivo with high-resolution magnetic resonance imaging. Stroke (2000) 112: 959-964
Fujimoto, J G et al. High resolution in vivo intra-arterial imaging with optical coherence tomography. Heart (1999) 82: 129-133.
Chapman, Trinh, Pfieffer, Chu and Lee. Angular Domain Imaging of Objects Within Highly Scattering Media using Silicon Micromachined Collimating Arrays. IEEE Journal of Selected Topics in Quantum Electronics. (2063) V9, No 2: 257-266.
Podoleanu, Review Article: Optical Coherence Tomography. British Journal of Radiology (2005) 78: 976-988

| | | |
|---|---|---|
| 4,953,539 | June 1990 | Nakamura et al |
| 6,010,445 | January 2000 | Armini et al |
| 6,134,003 | February 1996 | Tearney et al |
| 6,178,346 | January 2001 | Amundson and Hanlin |
| 6,529,770 | March 2003 | Grimlatov |
| 6,552,796 | March 2001 | DeBoer et al |
| 6,692,430 | February 2004 | Adler |

### FIGURES

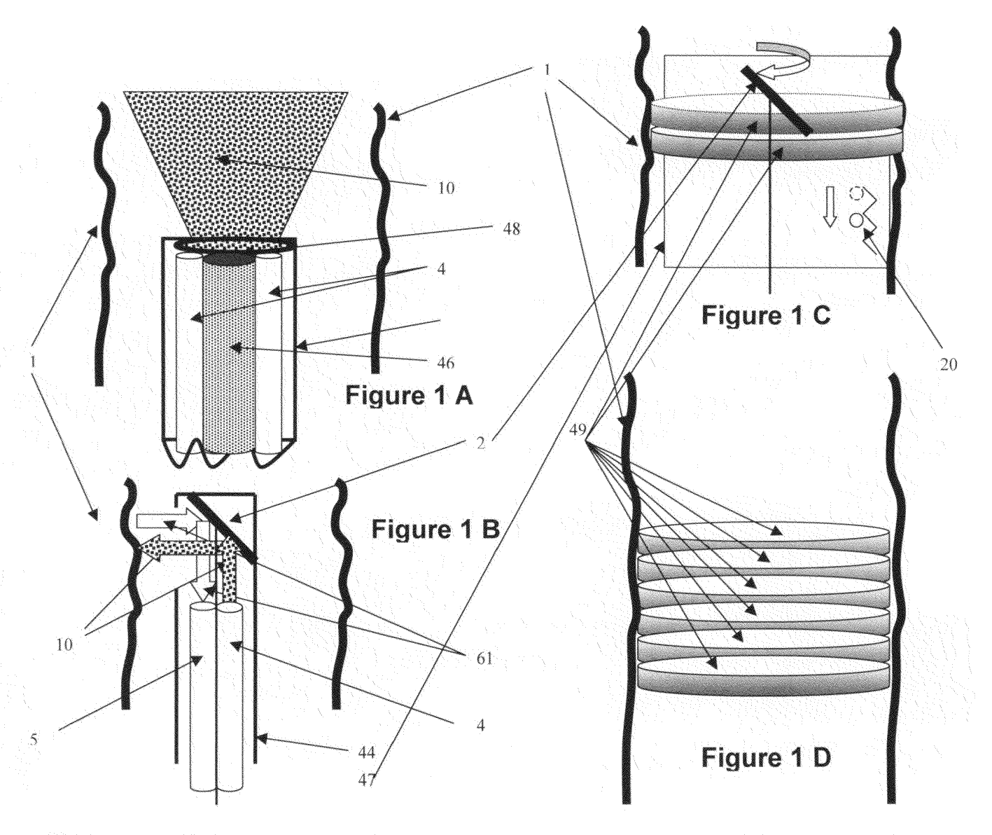

1. FIG. 1A depicts a conventional endoscope inside an artery. The endoscope emits light down the lumen of the artery. FIG. 1B depicts the invented scanner down the lumen of the artery. The scanner directs light nearly perpendicular to the arterial wall by reflecting the light off of a rotating mirror. The resultant image is a ring of the arterial wall. FIG. 1C shows the scanner translated along the vessel axis and the acquisition of two rings. FIG. 1D shows the acquisition of multiple rings.

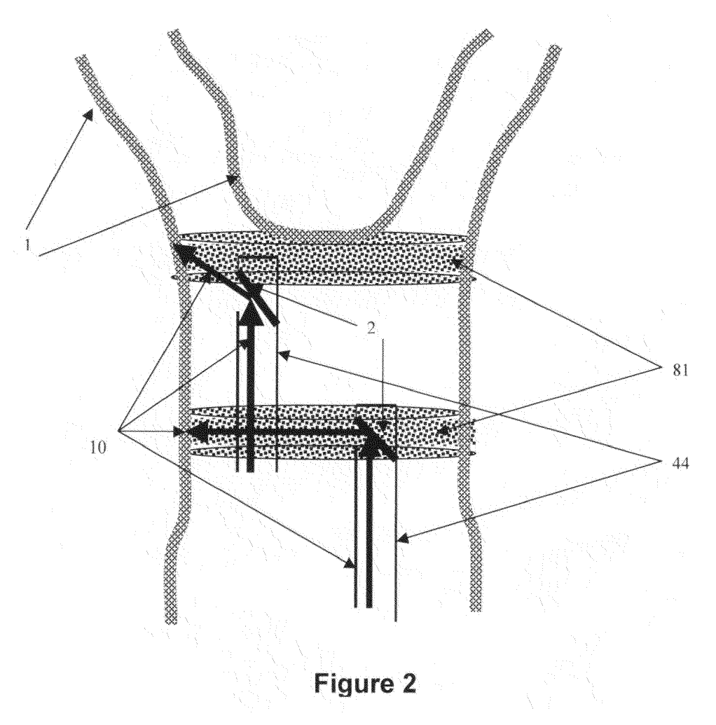

2. FIG. 2 depict two scanners inside an artery with a bifurcation. The lower scanner has a mirror reflecting light at a 90 degree angle. The upper scanner has a mirror reflecting light at an obtuse angle, creating an imaged ring ahead of the catheter.

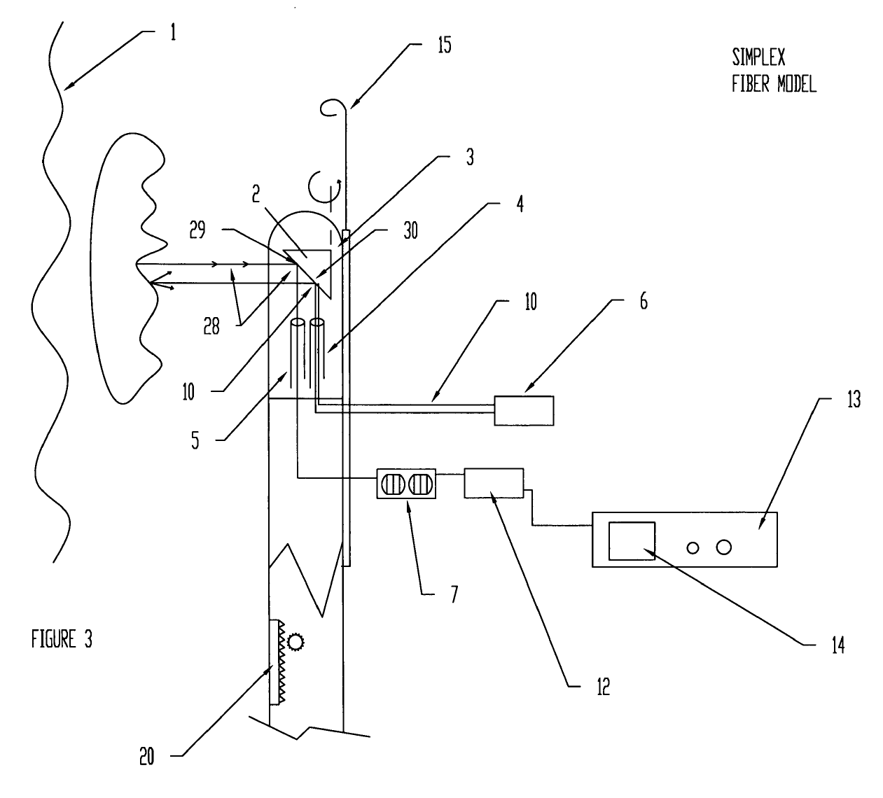

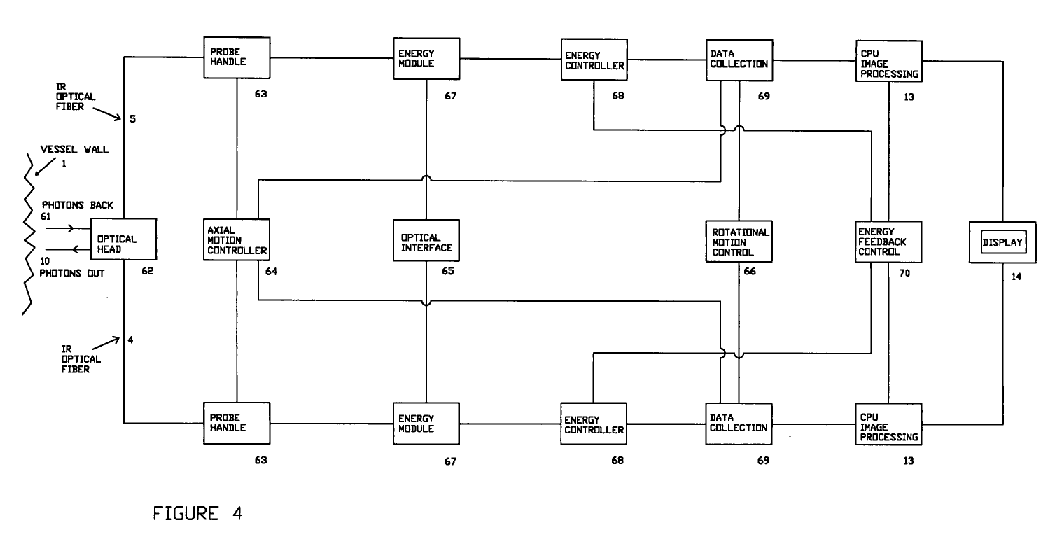

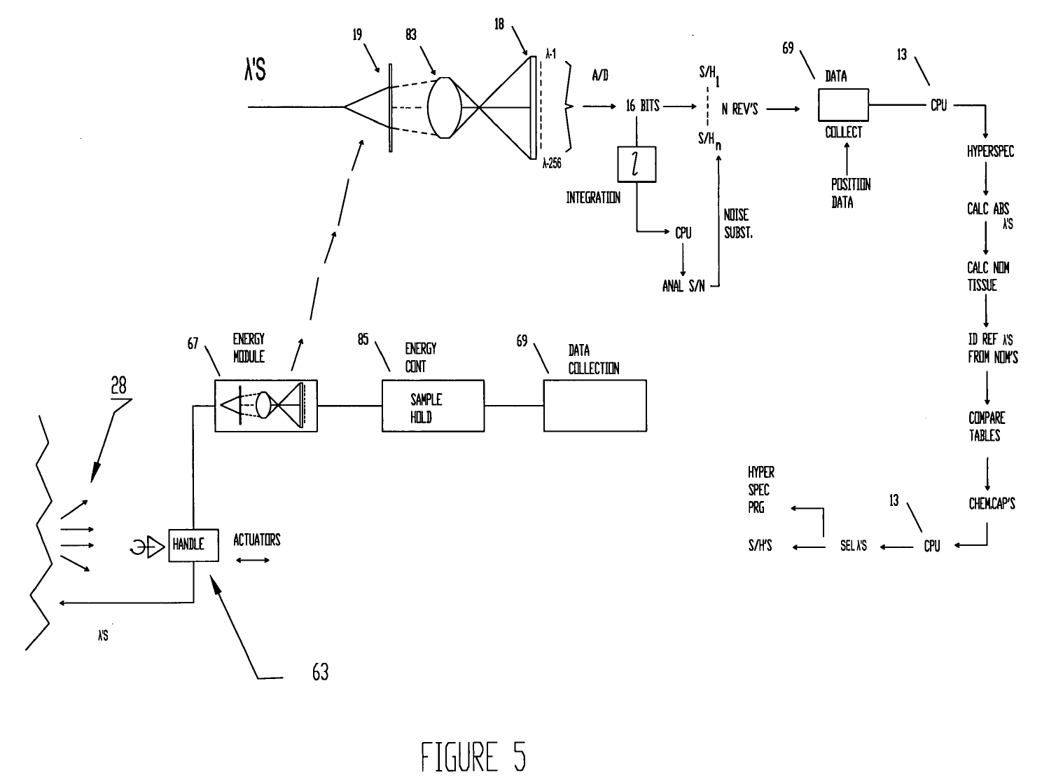

3. FIG. 3 depicts the scanner in an artery with a single receiving and emitting fibers.

4. FIG. 4 shows the block diagram of the system.

5. FIG. 5 is a schematic of the hyper-spectral imaging embodiment.

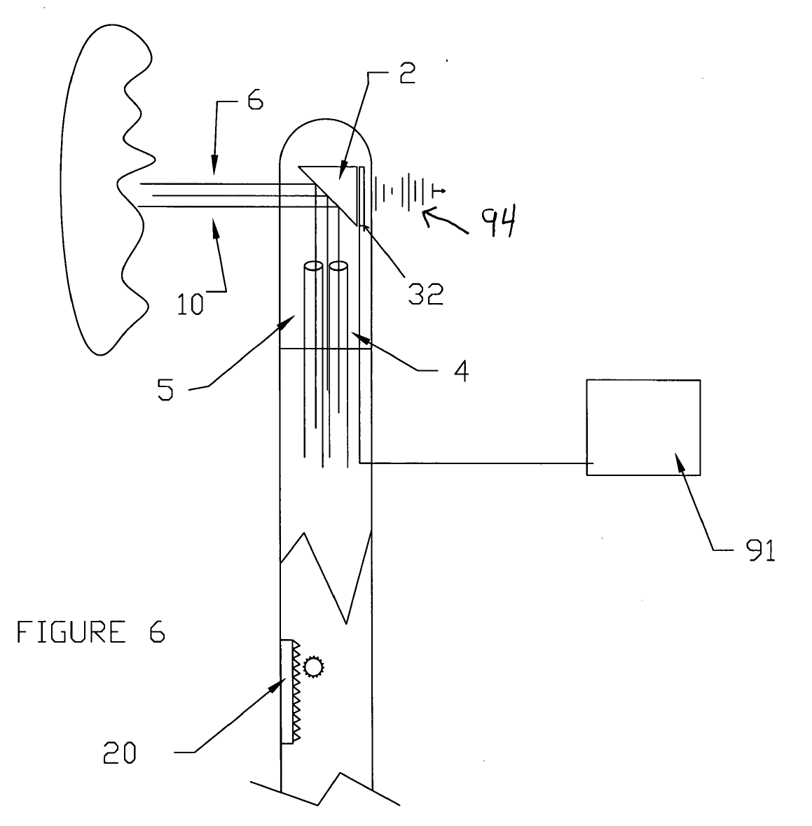

6. FIG. 6 is a drawing of the scanning light probe with a piezoelectric transducer for simultaneous IVUS images or ultrasound distance measuring

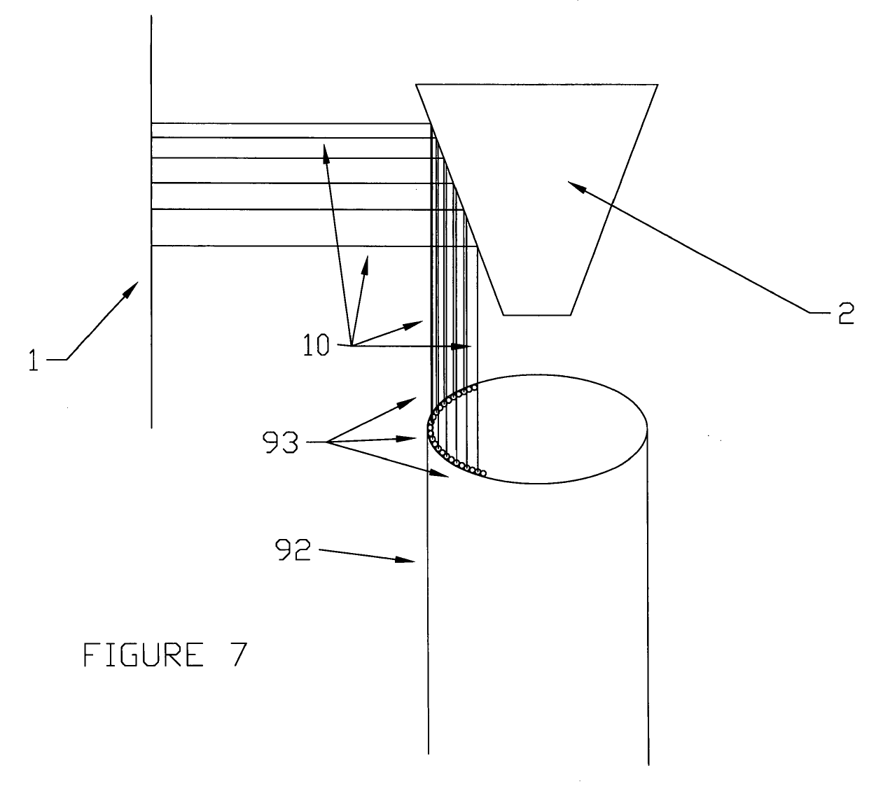

7. FIG. 7 depicts a multi-fiber embodiment of the light scanning imager

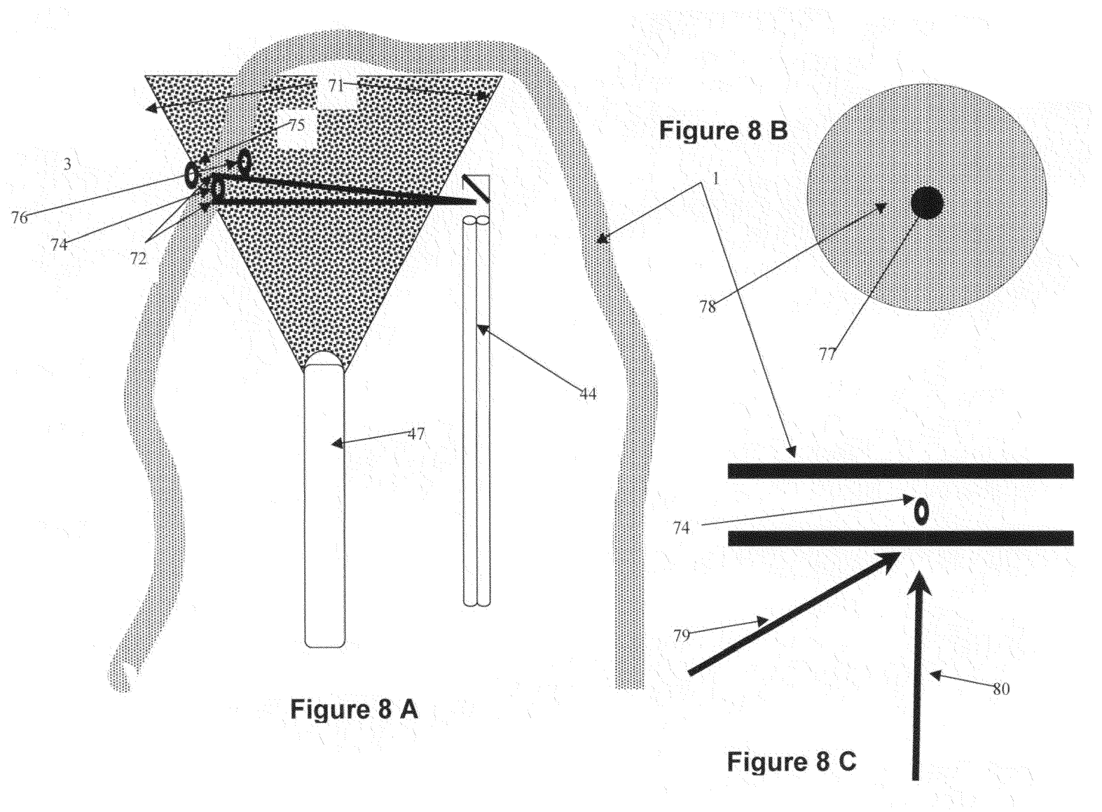

8. FIG. 8A shows both a traditional endoscope and the invented scanner inside of an elongate organ with cancerous cells on the surface, inside the organ and on the organ's surface. FIG. 8B shows the emission angle of a conventional endoscope and the invented scanner. FIG. 8C shows the incidence angle for a conventional endoscope and the invented scanner.

### BACKGROUND

### Light Imaging within the Body

Endoscopes are of interest in medicine because they provide a visual, light-based and minimally invasive means of exploring internal pathology. The endoscope provides the physician with a direct image “as if looking at it with his own eyes”. This has led to the field of minimally invasive surgery (MIS) where one channel in the body contains the endoscope and another contains a therapeutic tool which is directed to the pathology under view of the endoscope. The common visible light endoscope has limitations which are addressed by the infrared scanning imager discussed in this application. These are particularly profound in the examination of internal tubular structures, when the viewing field contains scattering particles or when the underlying chemistry or surface characteristics are required to ascertain the pathology. These include:

### 1. Non-Systematic Examination

The endoscope is manipulated by the physician who directs it to regions of interest. Frequently, sections of tissue are not imaged whatsoever.

### 2. Limited FOV

Commercial endoscopes have an FOV, ranging typically between 45-80 deg. Thus only a small sector of the 360 deg tubular anatomy is imaged at any given time. This prevents the accurate construction of pathology maps to aid the physician in his diagnosis and treatment plan

### 3. Forward Viewing

Most endoscopes view forward or are canted at a small angle. While this is traditional and the easiest device to construct optically, many applications such as tubular structures require viewing perpendicular to the endoscope axis. In this orientation, light is reflected near normal to the tissue surface which produces the highest contrast images.

### 4. Blinded by Scattering Media

Blood is the main scattering media. Visible endoscopes cannot image any meaningful distance through blood. Even structures with clear fluids with a small amount of blood (such as a bleeding stomach) cannot be imaged with visible endosocopy.

### 5. Unable to Make Spectrophotometric Measurements

The interesting part of the wavelength spectrum for recognizing biological and chemical entities is the region 1200-3600 nm. Biological and chemical entities have absorbance peaks in this region.

### 6. Cannot Measure Distances or Determine Object Size.

Light endoscopes cannot measure the distance to a structure surface or determine object size. Intravascular ultrasound (IVUS) is able to measure distance because the transit time of sound is easily measurable. The orders of magnitude higher velocity of light prevent this technique from being employed

### 7. Inability to Employ Signal Averaging Techniques

A powerful technique to improve resolution in light images is the use of signal averaging techniques. For this technique to be operable, multiple images of the exact same scene are acquired. An endoscope because of the motion caused by breathing, the heartbeat and manual manipulation by the physician, cannot acquire multiple images of the same scene.

### 8. Inability to View Fluorescent Dyes

A developing field is the introduction of a fluorescent dye applied systemically to the patient. The dye can preferentially attach itself in higher concentrations to pathological cells such as cancer or atherosclerotic cells. When these dyes are illuminated at a particular wavelength, they emit light at usually a higher wavelength. This small light emission can be detected by a viewing apparatus if the received signal is band-passed to be sensitive to a small wavelength region surrounding the emitted wavelength. A common dye is indocyanine green which needs to be stimulated at 760 nm and emits in the infrared at 810 nm. Conventional endoscopes usually neither apply intense light at the particular wavelength to fluoresce the molecule nor is it sensitive enough to view the emission.
Besides pathology, another field which uses fluorescent dyes is stem cell introduction. Since stem cells cannot be imaged in the body, a technique of attaching a fluorescent dye to the stem cell has been developed. This permits viewing the progression of stem cells introduced into an organ or structure.

### 9. Inability to Construct 3D Tomographic Images

Endoscopes only present a 2D image within a limited FOV. Since the exact location of the physician-manipulated endoscope is unknown, the collection of images cannot be stitched together by image processing programs. This would be desirable since it would allow the physician to look at it from different orientations as well as obtain a global sense of the pathology on the structure investigated

There are two important diseases, which involve tubular or elongate structures and pathology that is not discernable through other imaging means: atherosclerosis and cancer. Atherosclerosis is characterized by plaque ranging from soft (fatty plaque) to hard (calcified) or dangerous (vulnerable plaque) in the arterial walls. Direct imaging is impossible, unless the blood is evacuated and replaced with saline.

In the area of cancer imaging, a visible endoscope does not systematically view the entire organ surface. Rather, it is left to the physician to explore all areas of the structure. Moreover, depending on the angle of the endoscope to the tissue, the reflected image detection will vary in contrast because of the different light conditions, further complicating the cancer detection. At the conclusion of the endoscopic examination, no “cancer map” of the structure is available.

Since visible light only penetrates tissue modestly, only cancerous cells on the structure surface will be imaged. Cancerous cells in the wall of the structure can go undetected.

An important advance in oncology is using fluorescent dyes to highlight cancerous cells. This is accomplished by infusing a fluorescent dye either systemically or locally within the structure. Cancerous cells preferentially absorb the dye. When light of a particular wavelength is illuminated on the structure, the dye fluoresces weakly at a higher wavelength and the cancerous cells are revealed. Endoscopes have been modified to detect certain visible fluorescent dyes. They cannot detect the infrared dyes such as indocyanine green, which emits at 810 nm. Furthermore, they do not illuminate the structure uniformly, reducing the chance of activating fluorophores attached to cancerous cells within the structure surface.

Heart Disease

The latest statistics from the American Heart Association are disturbing: In 2004, an estimated 865 000 Americans will develop a new acute coronary syndrome. Another 700 000 will have a stroke. Unfortunately, the contribution of percutaneous coronary intervention (PCI) to prevent such catastrophic outcomes has been limited. To date, intervention cardiologists have been constrained to the treatment of obstructive atherosclerosis disease in certain regions of the coronary tree and arteries in the neck and brain. This approach has a clear benefit in reducing ischemia and symptoms but minimal direct impact on patients' survival.

Underlying the lack of survival-improving strategies is the inability to image the arterial wall. Today, the vessel wall is mostly imaged through indirect methods, such as X-ray imaging of radio-opaque dye infusion and intraluminal ultrasound (IVUS). Arterial narrowing or constrictions are imaged with both technologies. Needless to say, they do not provide a direct light-image of the vascular wall.

In the 1980's, light endoscopes called angioscopes were developed. These devices displaced blood from the viewing field by injecting saline. This would permit an episodic clear field before the saline is replaced by blood. Despite initial enthusiasm, it is rarely used today in the US or Europe, because the replacement of blood with saline was tedious and potentially dangerous. Additionally, a technology based on interferometric principles (OCT) has been developed, but shares with angiosocopy the requirement of replacing the blood field with saline solution.

In the 1990's, a number of studies, principally from visual examination of post-mortem specimens have determined that the major culprit involving serious heart attacks and strokes arise from a certain kind of plaque with a liquid core, much like a blister on the skin.

Vulnerable Plaque

It is now recognized in the cardiology community that most serious heart attacks and strokes are due to a particular type of plaque formation called “vulnerable plaque”. Vulnerable plaque consists of a thin fibrous capsule containing a gelatinous fluid consisting of lipids and blood cells. When it ruptures (usually due to emotional or physical stresses), the released fluid can cause massive coagulation. If a vulnerable plaque ruptures in the coronary arteries, it can lead to a massive heart attack; in the carotids, a massive stroke. “The rupture of a plaque will be the cause of death of about half of all of us in the United States,” says Dr. Steven Nissen of the Cleveland Clinic in a 1999 Associated press article by Daniel Haney. “Understanding why they rupture is probably the most important question today in cardiology and even the most important question in all the country.” A recent article in Stroke arrives at a similar conclusion “Cardiovascular disease is the leading cause of death in the United States and >70% of these deaths are related to atherosclerosis . . . >75% of the major coronary events were precipitated by atherosclerotic plaque rupture”

Current Imaging of Vulnerable Plaque
Vulnerable plaque is currently not diagnosable. “We have no tools at the moment to recognize which sites are vulnerable. It's guesswork,” says Dr. Renu Virmani of the Armed Forces Institute of Pathology in Washington, D.C.¹ “Characterizing the nature of the fibrous cap that overlies lipid-rich plaque core may be more productive. For example, a thinned fibrous cap may be more prone to rupture. Defining the surface morphology of the lesion may also be important. In a review of the first 500 patients enrolled in the North American Symptomatic Carotid Endarterectomy Trial, Streifer found that the sensitivity and specificity of detecting ulcerated plaques were only 34.9% and 74.1% respectively.”

“Characterization of the fibrous cap and plaque surface morphology remains a significant challenge for ultrasound and MRI. As with angiography, physical restrictions limit the number of views obtainable with transcutaneous B-mode ultrasonography . . . “In conclusion, this study found that intraplaque hemorrage, the lipid core, necrotic core, and calcifications are commonly found in highly stenotic carotid plaques. Furthermore, the volumes of these materials are similar in plaques removed from asymptomatic and symptomatic individuals. From an imaging perspective, it is unlikely that identification of these plaque features will distinguish severe carotid stenosis that are higher risk for developing ischemic neurological symptoms.”

Hatsukami reached a similar conclusion in his study of morphology of carotid artery plaque “advancements in ultrasound and MRI technology continually improve the prospects for precise quantitative imaging of arterial wall pathology. In this histological study, the volumes of the lipid core, intraplaque hemorrhage and calcification failed to discriminate thrombus removed from patients who had clinically recognizable ischemic neurological events from those who were asymptomatic. These findings suggest that in highly stenosed plaques, identification and quantification of these plaques by MRI or ultrasound will be unlikely to distinguish lesions that are at high risk of ischemic events from those that are likely to remain clinically silent. Characterizing the nature of the fibrous cap that overlies lipid-rich plaque may be more productive.”

Importance of Surface Features

Surface features, such as ulcerations cannot be seen with conventional technologies because of the inherent low-resolution of these technologies (X-ray or sound wave), especially with respect to soft tissue. In a 1999 Heart article, J. G. Fujimoto states³ “a need exists in at least two areas of cardiology for an imaging technology capable of defining arterial structure on a micron scale. These areas are the identification of high-risk coronary arteries and guidance of interventional procedures (for example provisional stenting). The rupture of small, thin-walled, lipid-filled plaques in the coronary arteries has now been established as a critical mechanism, resulting in acute coronary syndromes. Current imaging technologies cannot reliably identify these lesions before rupture, predominantly because of limitations in resolution. Similarly, high-resolution real time imaging of plaque microstructure will likely also be beneficial in guiding coronary interventional procedures such as directional and rotational atherectomy. Although these catheter-based interventions are microsurgical procedures, removing tissue of only a few millimeters in depth, they are primarily guided by fluoroscopy, which has a resolution in the range of 500 microns during cardiac motion. IVUS, the current clinical technology with the highest resolution (approximately 100 microns) has been applied to both the identification of high-risk plaques and the guidance of interventional procedures. However, the reproducible identification of vulnerable lesions has not been achieved and its utility for guiding interventional procedures may be limited to stent placement”.

Optical Methodologies of Imaging Vascular Tissue

Optical vascular imaging technologies include those, which image vascular wall by replacing the blood with saline which can image vascular wall through an intervening blood field. Those requiring blood-field replacement with saline include angioscopy, near-infrared spectroscopy (NIR) such as practiced in U.S. Pat. Nos. 6,873,868, 6,654,630 and optical coherence tomography (U.S. Pat. No. 6,552,796) Technologies which can image through blood are described in patents by Grimblatov (U.S. Pat. No. 6,529,770 B1) Amundson & Hanlin (U.S. Pat. No. 6,178,346) Adler (U.S. Pat. No. 6,692,430)

Optical Methodologies of Imaging Vascular Tissue by Saline Replacement

Angioscopy systems have been manufactured over the last twenty years. While the optics are identical to conventional endoscopes, they also include a means for replacing blood with saline. The means is typically an occluding balloon and a port for saline infusion. Although only rarely used today, angioscopy has contributed to the understanding of vulnerable plaque. Various Japanese investigators have recently observed a glistening yellow appearance of vulnerable plaque using angioscopy. Even these more recent observations are confounded by blood entering the saline field and by the forward-viewing nature of he angioscope. These limitations prevent detailed direct surface-viewing of plaque.

Unlike angiosopy, NIR and OCT are not direct-imaging technologies. Direct-imaging is reflected light scattered off of the object of interest and received by an eye or camera. NIR and OCT obtain their imaging data indirectly using either an interferometric technique (OCT) or a spectrophotometric technique (NIR). The sensitivity of these measuring techniques prevents application in blood fields; the scattering of light by red blood cells greatly increases noise, which swamps the small real signal amplitude.

Optical Methodologies of Imaging Vascular Tissue Through Blood

Grimblatov (U.S. Pat. No. 6,529,770 B1) Amundson & Hanlin (U.S. Pat. No. 6,178,346) and Adler (U.S. Pat. No. 6,692,430) describe different means of imaging through a blood field. When blood cells (principally red blood cells) are present between the imager and the target, the signal to noise ratio reduces dramatically. Most light reflected or scattered from a vascular target undergoes multiple scatter events from red blood cells, thereby greatly decreasing the signal to noise ratio and lowering contrast. This is compounded in the infrared by increases in optical absorption, further decreasing the signal component. The challenge is to increase the signal to noise ratio when imaging structures through blood.

The above three patents have different means of increasing the signal to noise ratio. Grimblatov (U.S. Pat. No. 6,529,770 B1) relies on irradiating vascular wall with particular infrared wavelengths in the band around 1.0-1.2 microns. These wavelengths have maximum optical path length coupled with various means of subtracting out the noise to achieve a higher S/N level. These means include subtraction of certain areas of the image, subtraction of a secondary wavelength which is opaque to the vessel wall, subtraction of low-amplitude signals, which is also opaque to vessel wall and others.

Amundson & Hanlin (U.S. Pat. No. 6,178,346) use a laser diode with an infrared wavelength set at an optical absorption minimum and high enough in the infrared spectrum to reduce scattering. It is established in optical science by G. Mie in the early 1900's that infrared light penetrates suspended particles to a greater degree as wavelength increases. This concept has been used for decades in imaging through fog. Unfortunately, in a liquid medium, infrared light is no longer transparent but absorptive to various degrees, depending on wavelength. At wavelengths high enough to substantially reduce scattering, the water component of blood becomes very absorptive of infrared light. This significantly reduces the reflection of the imaged object, worsening the S/N ratio. As in Grimblatov, various methods of subtracting out background noise needed to be employed to compensate for the small “true” signal brought about by the infrared-absorbing medium.

A system incorporating this invention was produced by the company CardioOptics Inc. for use in identifying the coronary sinus in biventricular pacing implantations. The system employed a forward-viewing catheter with an 80 degree field of view (FOV). A variety of monochromatic wavelengths with minimal absorption/low scattering characteristics were tested including 1.0 microns, 1.3 microns, 1.55 microns and 1.6 microns. An FDA-approved human clinical trial was conducted using a wavelength of 1.55 microns. Problems with a monochromatic wavelength include speckle production in the image and insensitivity to the multiple “signature” absorbances of biological material.

Despite the selection of ideal wavelengths to maximize the S/N ratio, considerable noise and low signal amplitudes still prevailed. Depending on the target distance, the noise was many times greater than the signal. Subtraction of the noise would often lead to false artifacts appearing as holes or bright spots. This confounded the physician who was unsure if he was seeing a feature and not an artifact produced by the noise-subtraction algorithm. Despite these limitations, structures could be imaged episodically about a half a millimeter from the catheter. Greater viewing distances (around a millimeter) were possible if the catheter axis was normal to the structure and if the structure had sharp features.

Additionally, the FOV of 80 degrees combined with the short viewing distances proved to be inadequate to evaluate surfaces in the heart or the vasculature. In the heart, the catheter required physicians to frequently manipulate the catheter to image the desired structure. The short viewing distance required the physician to place the catheter tip within about a millimeter from the surface. These tasks were especially difficult in the beating heart, which often displaced the imaging catheter tip with each heartbeat. This catheter was also placed in the vasculature in patients and animals. In the vasculature, the forward-viewing orientation of the catheter relegated the vessel wall to the periphery of the image where the S/N was the worst, resulting in distorted or insufficient images.

Adler (U.S. Pat. No. 6,692,430) teaches a means of providing images of the vessel wall by locating an image sensor in a non-perpendicular orientation. This is an improvement to the forward-viewing endoscopes of Amundson and Grimblatov. No method is given to see through blood, other than suggesting an appropriate wavelength to see through blood. This approach is limited to image sensors which can be miniaturized: CCD or CMOS sensors. These sensors are only capable of imaging to 1.0 micron (1000 nm).

Nonetheless, for vessel-wall viewing through blood, the non-perpendicular orientation is useful since it will image more vessel wall then a forward-viewing catheter. As in the Amundson and Grimblatov patents, the receiving optics has a fixed FOV, limiting viewing to sections of the vascular wall.

The three patents Grimblatov (U.S. Pat. No. 6,529,770 B1), Amundson & Hanlin (U.S. Pat. No. 6,178,346) and Adler (U.S. Pat. No. 6,692,430) all share the methodology of employing a particular wavelength or several wavelengths which make blood less opaque. This is combined, in the case of Grimblatov and Amundson, with subtraction means of reducing the noise created by multiply-scattered diffuse photons.

Experiments with multiple wavelengths and subtraction techniques strongly suggest that the noise is too great to yield faithful images of the underlying anatomical structures through blood for any meaningful distance. Judicious wavelength choice and noise subtraction algorithms are not the answer.

Moreover, the endoscope-nature of Grimblatov (U.S. Pat. No. 6,529,770 B1) Amundson & Hanlin (U.S. Pat. No. 6,178,346) and Adler (U.S. Pat. No. 6,692,430) significantly limit its usefulness in the vasculature. The forward-viewing catheters of Grimblatov (U.S. Pat. No. 6,529,770 B1) and Amundson & Hanlin (U.S. Pat. No. 6,178,346) only image the vascular wall on the periphery of the FOV where the S/N is the worst. Furthermore, to image vascular wall, light has to backscattered 180 deg from the incident light beam to register an image of the wall.

Adler (U.S. Pat. No. 6,692,430) directs the light perpendicular to the vascular wall, where the reflected light will be at a maximum. However, only a sector of the vascular wall is imaged.

Cancer

Endoscopes have been used for decades to detect cancerous cells in many internal structures. These structures include, but are not limited to the esophagus, colon, stomach, fallopian tubes, lungs, kidneys, bladder, trachea etc.

A common procedure is to insert a visible light endoscope into the internal organ and observe the surface tissue, looking for the characteristic signs of cancerous cells. Nakamura (U.S. Pat. No. 4,953,539) describes an infrared endoscope inserted into the patient with external infrared illumination to image cancer on the exterior surface of an organ, such as the bladder. Infrared light is more useful in cancer detection because infrared light penetrates tissue more deeply than visible light, permitting cancerous cells within and on the exterior of the tubular structure to be detected. Moreover, structures can be imaged through murky fluids such as blood, transparent fluids made murky by blood, cerebrospinal fluid and any murky fluid made opaque by the presence of biological cells.

Another advantage of infrared light is that the infrared spectrum from 1300-3600 nm contains absorbance maxima for many biological components. Cancer cells are of a different chemical composition than normal cells. The presence of cancerous cells can be imaged by overlaying on the image obtained from polychromatic light, an image illuminated by a wavelength at an absorbance peak corresponding to a prominent chemical entity of the cancerous cells. This can be accomplished using a band-passed polychromatic source or a monochromatic laser or LED. Even structures without fluids could be examined in the infrared using the diffractive element and the methods above. In the wavelength regions where cancer absorbance peaks are present, the scanning device of this patent application could have an algorithm to emphasize the cancer-absorbance-peak wavelength by subtracting out the background produced by other wavelengths.

Also, fluorescent dyes, such as indocyanine green, are used as light emitting markers of cancerous cells. The dye is administered to the patient and when directly illuminated at a certain wavelength (760 nm for indocyanine green) it emits light at a higher wavelength (810 nm for indocyanine green). Modified endoscopes have been constructed which emit light at 760 nm and are bandpassed to accept reflected light in a narrow wavelength region centered at 810 nm and transmit it to a CCD camera which is sensitive up to 1000 nm—higher than the human eye.

The disadvantages of detecting cancer with an endoscope inside an internal structure are the following:

### 1. Non-Systematic Examination

The endoscope is manipulated by the physician who directs it to try to view the entire structure surface in the search for cancerous cells. No comprehensive “cancer map” can be constructed to aid the physician in his diagnosis and treatment plan—just a collection of episodic images Frequently, sections of tissue are not imaged whatsoever.

### 2. Limited FOV

Commercial endoscopes have an FOV ranging typically between 45-80 deg. Thus only a small sector of the 360 deg anatomy of many elongate organs is imaged at any given time.

### 3. Forward Viewing

Most endoscopes view forward or are canted at a small angle. While this is traditional and the easiest device to construct optically, many applications such as elongate organs require viewing perpendicular to the endoscope axis. In this orientation, light is reflected near normal to the tissue surface which produces the highest contrast images.

### 4. Blinded by Scattering Media

Blood is the main scattering media. Visible endoscopes cannot image any meaningful distance through blood. Even structures with clear fluids with a small amount of blood (such as a bleeding tumor) cannot be imaged with visible endosocopy.

### 5. Unable to Make Spectrophotometric Measurements

The interesting part of the wavelength spectrum for recognizing biological and chemical entities is the region 1200-3500 nm. Biological and chemical entities have absorbance peaks in this region and can be distinguished from normal tissue.

### 6. Cannot Measure Distances or Determine Object Size.

Light endoscopes cannot measure the distance to a structure surface or determine object size.

### 7. Inability to Employ Signal Averaging Techniques

Cancer cells can be difficult to distinguish from normal cells. Moreover, they can be present within tissue or on the organ interior instead of the inside surface, further reducing the reflected light intensity. A powerful technique to improve resolution in light images is the use of signal averaging techniques. For this technique to be operable, multiple images of the exact same scene are acquired. An endoscope because of the motion caused by breathing, the heartbeat and manual manipulation by the physician, cannot acquire multiple images of the same scene.

### 8. Inability to View Fluorescent Dyes

A developing field is the introduction of a fluorescent dye applied systemically to the patient. The dye can preferentially attach itself in higher concentrations to pathological cells such as cancer. A common dye is indocyanine green which needs to be stimulated at 760 nm and emits in the infrared at 810 nm. Conventional endoscopes usually neither apply intense light at the particular wavelength to fluoresce the molecule nor is it sensitive enough to view the emission. Endoscopes, which have been modified to emit light at 760 nm and receive light in the narrow region around 810 nm, still have limitations:
1. The large FOV does not concentrate the light sufficiently to fluoresce all of the indocyanine green molecules. Those attached to cancerous cells within the structure wall or on its exterior surface are unlikely to be detected.
2. Light is directed at various angles with respect to the vessel surface because the endoscope is manually manipulated. Both the incident and received reflected signal deteriorate when viewed non-normal to the surface.

### 9. Inability to Construct 3D Tomographic Images

Endoscopes only present a 2D image within a limited FOV. Since the exact location of the physician-manipulated endoscope is unknown, the collection of images cannot be stitched together by image processing programs. This would be desirable since it would allow the physician to look at it from different orientations as well as obtain a global and temporal sense of the cancer pathology to aid the physician in his diagnosis and treatment plan

### Optical Theory

### Imaging Through Murky Media

Endoscopes including the infrared endoscopes of Amundson and Hanlin (U.S. Pat. No. 6,178,346) and Grimlatov (U.S. Pat. No. 6,529,770 B1) cannot image structures through flowing blood with any meaningful distance by using a specific infrared wavelength. These methodologies result in a low signal to noise ratio. Subtraction of the large noise element can lead to false and unstable images.

Ballistic, Snake and Diffuse Photons

Another way of envisaging the problem of light passing through murky media, such as blood, is to focus on the scattering properties of the individual photons. Based on optical principles, photons passing through a turbid media can be multiply-scattered (diffuse photons), minimally scattered (snake photons) or unscattered (ballistic photons). Depending on the scattering medium and other factors, the vast majority of photons are diffuse, orders of magnitude less are snake and a many orders of magnitude are ballistic. The challenge of detecting objects embedded in murky media using photon theory is to collect ballistic and snake photons, which contain imaging information and significantly reduce the diffuse photons since they contain no imaging information because of their multi-scattering and instead create noise. Snake and ballistic photons are present at any wavelength. The best wavelengths for detection of snake and ballistic photons are those, which have low scattering and low absorption. In the middle of the visible spectrum (500-600 nm) hemoglobin has two absorption peaks. From about 600-1100 nm absorption is low and scattering is reduced. At around 1300 nm scattering is reduced but absorption is significantly higher. Additionally, detection of these light wavelengths requires an infrared camera. CCD cameras, which are much cheaper and more sensitive than infrared cameras cannot detect wavelengths above about 1000 nm. Higher up in the infrared in the region 1550-1850 nm, scattering is reduced some from scattering at 1300 nm, but absorption becomes very high. It also requires an infrared camera.

Methodologies, which preferentially collect snake and ballistic photons and filter out diffuse photons include the following:

1. Time-Gated Techniques.
Ballistic photons undergo a direct path, so they arrive at the detector first. By time-gating with femtosecond laser pulses, only the initial photons are captured. The drawback is the complex instrumentation required.

2. Coherence Techniques (OCT)
With the use of an interferometer and a reference beam, the ballistic and snake photons can be separated from the diffuse using the principle of coherence. It directs a reference laser beam to a detector to measure only photons in phase with the source (Podoleanu). It does not function in a blood field do to excessive noise.

3. Polarization Techniques
Placing a polarizer over the transmitting and receiving optics polarizes the outgoing light. Receiving light through the polarizer only permits light transmission of photons which had their polarization not altered by multiple scattering events. The technique requires more light energy to compensate for the filtering effect of the polarizer.

4. Angular Domain Techniques
The more a photon is scattered, the more it starts to deviate from the path of the incoming beam. Ballistic and snake photons can be preferentially sensed by drastically narrowing the angle of acceptance of the receiving optics. This has been accomplished with collimated sources and detectors but has not been implemented with optical fibers because the numerical apertures (N/A's) have been too large (Chapman et al).

Recent Optical Innovations

Recently, optical flexible fiber manufacturing has perfected fibers from doped silica or hollow fibers of very low NA. Previously, fibers had N/A's no lower than about 0.2. Today, fibers with N/A's lower than 0.1 are being routinely constructed by fiber manufacturers.

Also, laser diodes capable of generating multiple wavelengths have been developed and called polychromatic laser diodes. Previously, laser diodes were of a single wavelength. This development permits the use of high-energy lasers spanning the near infrared spectrum. Biological substances have absorption peaks in the region 1100-3600 nm. Exposing tissue to wavelengths around these peaks creates a darkening of the image where the particular chemical moiety is present.

Another means of producing polychromatic laser light in a fiber is the use of doped illumination fibers. These fibers are chemically modified, permitting certain wavelengths to be preferentially transmitted and creating a polychromatic source. This method of producing polychromatic laser light uses doped fibers that are pumped by several monochromatic sources to produce polychromatic wavelengths.

### SUMMARY OF THE INVENTION

The embodiments of this invention describe the detection of atherosclerotic plaque or cancer cells by a light probe inside a blood vessel or internal to an elongate organ. The light probe is a scanner which projects light circumferentially, approximately perpendicular to the vessel/organ surface light image and receives light with a low-angle acceptance criteria. The combination of projecting light nearly perpendicular to the vessel/organ surface and receiving light with low-angle acceptance criteria permits the collection of a sufficient number of non-scattered and minimally scattered photons to create a circumferential, ring image of the vessel/organ surface, even through scattering media, such as flowing blood.

FIGS. 1A-D illustrates the scanning concept. In FIG. 1A, a conventional endoscope (47)) is depicted inside an elongate structure. Light (10) is projected at an FOV of 60-80 degrees, principally down the lumen of the structure. The walls (1) of the elongate structure are only obliquely illuminated, resulting in low-contrast images on the image periphery, which also compromises imaging accuracy. The field is illuminated by two optical fibers (4) and the reflected light received by a multi-fiber imaging bundle (46) and transmitted to a camera. FIGS. 1B-D illustrates the principles of the light scanner (44). Light (10) exits the distal end of the optical fiber (4) where it is directed at a mirror (2) and deflected about 90 degreed from the probe or vessel axis. The light (10) reflects off the wall (1) of the elongate structure, reflects off the mirror (2) and is received by an optical fiber (4) and transmitted to a detector. The detector amplitude is recorded in a computer. As seen in FIG. 1C, the mirror (2) rotates about the probe axis, illuminating a ring (49) on the vessel surface. The reflected light amplitudes in all mirror positions are stored in the computer. After one revolution a circumferential ring of the vessel/elongate organ surface is obtained. As seen in FIGS. 1C and 1D, multiple rings (4) are created by translating the probe using a translator element, such as a worm gear (20). Another means of translation is manual withdrawal with a translation detector in the probe handle to measure the translation distance. The resultant N rings obtained over the translation distance are concatenated and filtered to create a panoramic image of the entire vessel wall over the translation distance. Highest contrast images occur when the emitting and receiving optics are both near-normal to the surface being viewed. In one embodiment, vessel wall is imaged through flowing blood by employing a scanning mechanism using one emitting and one receiving fiber, whereby light is directed at a spinning mirror, approximately normal to the vessel or elongate organ surface. The light is reflected circumferentially around the vessel or elongate organ surface as the mirror rotates and received by a low-numerical aperture (NA) fiber, which transmits it to a light detector, thereby generating a set of light amplitudes circumferentially around the vessel/elongate organ surface. Multiple rings are acquired by translating the probe within the vessel/elongate organ. A computer subtracts background noise and concatenates the rings to create a panoramic image on the computer monitor of the vessel or elongate organ surface over the length of the translation.

In another embodiment, adding a piezoelectric transducer in proximity to the distal ends of the fibers permits simultaneous ultrasound and light images to be created. Since ultrasound permits distance measurement, 3D, tomographic images are also created. Adding a polarizer over the emitting and receiving waveguides further expands the viewing distance.

In another embodiment, multiple low-NA receiving fibers are employed and the light is transmitted to a linear array detector.

In another embodiment, instead of using low-NA receiving fibers, a telecentric lens creates the low-angle acceptance condition and transmits the light to an area array camera. In another embodiment, the chemical composition of the vessel surface or elongate organ can be determined by transmitting the backscattered light to a dispersive element, which divides the light into multiple wavelength regions. The dispersive element is focused unto a linear or area array camera to create an image of the vessel/organ surface with wavelength-selectable highlights.

In another disclosure, the identification of fluoroscopic dye emissions is optimized by directing high-intensity light with small emission angle near-normal to the vessel/elongate organ and receiving the fluorescent emission with a low-NA fiber.

The present invention provides methods and means and apparatus to sharply filter highly scattered diffuse photons using low-NA fibers to render a 360 degree, 2D or 3D direct image of vessel wall surface over several centimeters. A series of experiments in flowing blood conditions demonstrate that a field of view (FOV) of 30 degrees (acceptance angle of 15 degrees) is required to image the vessel wall at 3 mm—the minimum distance required in a coronary artery device. Greater viewing distance and image clarity are achieved with FOV's substantially lower. Often, FOV's of 1 degree or less will be required to produce fractional angular image components of the 360-degree scans, that when taken as a whole are high quality images by filtering the highly scattered or diffuse photons with low-angle acceptance criteria, such as a low-NA fiber.

In the case of a FOV of 1 degree, 360 images will be accumulated as the mirror makes a complete revolution. The sensor is then translated to the next position and the cycle repeated. A series of ring images are thus created. These are then concatenated to create the entire vessel wall over 1-2 cm. Making multiple passes and averaging the signals can achieve high resolution or greater viewing distance. Adding a polarizer over the emitting and receiving waveguides further improves the viewing distance

The present invention with either diffractive, band pass filters, or holographic elements or doped fibers provide methods and means of imaging the chemical composition of plaque, differentiating plaque according to its cholesterol, calcium and lipid content. Since vulnerable plaque consists of a lipid pool covered by a thin (˜40 nm) membrane, The lipid pool, which is penetrated by infrared light, would be more absorptive at wavelengths near the absorbance peaks for lipids (between 1700-3600 nm). The wavelength separating elements produce multiple images in each wavelength of interest. The 2D and 3D images mentioned above can be compared with known image data using wavelengths known to be near absorption peaks for a particular chemical entity. The computer comparison can aid in determining the chemical composition present. Much of the chemical composition of plaques and pathologies are well known and cataloged. Once potential vulnerable plaque is identified, high-resolution light images of the vulnerable plaque can be achieved using the techniques described above and can be augmented using multiple scans, which permits signal averaging. High resolution light images permits classification of vulnerable plaque sites according to their surface characteristics and ultimately in the identification of vulnerable plaque most likely too burst and cause a fatal heart attack or stroke. For example, vulnerable plaque about to burst is of higher pressure which alters the shape of the vulnerable plaque cap.

In addition to plaque detection by chemical analysis, cancer also has different biological components and can also identified by its chemical signature.

Cancer detection is improved beyond endoscopic examination because the light is directed and received nearly perpendicular to the surface in a elongate structure. Even in a structure marginally elongate, such as the stomach or esophagus, the incident light is usually +/−30 deg from normal incidence—a situation, which enhances reflected light detection.

The present invention also provides an improved means of sensing fluorescent dyes, both visible and infrared dyes. The near-normal incidence of small-FOV light permits high light fluxes at the structure surface and increases the likelihood of sensing fluorescent dye molecules attached to cells within or the structure surface exterior. For example, if the cancer cells (FIG. 2A, 74,75,76) had a fluorescent molecule attached to them, the cancer cell on the organ exterior (76) would have a greater chance of being excited since the light flux would be orders of magnitude higher at the surface. As the light is scattered and absorbed by the tissue wall (70), enough light may be present to fluoresce the molecule on the surface exterior.

The fluorescence from a fluorescent molecule on the surface exterior passes through the organ surface, where it may be too faint for detection by the sensor. An advantage of the scanner is multiple rotations can be executed, allowing the accumulation of faint fluorescence to create a larger signal viewable on a computer monitor. Periodic examinations of the same segment of the structure can be compared to reveal the progression of fluorescent-tagged cells such as fluorescent-dye-tagged stem cells.

The present invention also provides methods and means of sizing the entire 1-2 cm vessel section using an ultrasound transducer couple to the receiving fiber(s).

The present invention also provides methods and means of coupling the infrared image to a ultrasonic IVUS image to provide both surface detail (infrared image) and intra-vessel wall images (IVUS).

The present invention also provides methods and means of imaging the surface features together with features inside the arterial wall, such as the lipid pool in vulnerable plaque.

The present invention also provides methods and means of distinguishing various types of plaque, from calcified to fibrous to vulnerable plaque based on a combination of surface features and chemical analysis.

This invention provides methods and means of utilizing polychromatic infrared light, filtered incandescent, polychromatic laser diodes, diffractive elements or doped fibers to collect high-resolution infrared images using a multitude of wavelengths. This allows for chemical identification of tissue with different optical absorption properties. Examples include vulnerable plaque and cancerous tumors or lesions.

### DETAILED DESCRIPTION OF THE INVENTION

### Experimental Data

Background

The inventors have experimented with infrared imaging through flowing blood using a traditional endoscope over the period 1997-present. This included initial in vitro experiments, open-heart rigid endoscope experiments, animal experiments with a percutaneous endoscope with an FOV of 80 degrees, and finally human clinical experiences. In an effort to view longer distances through blood, the inventors experimented with a smaller FOV from 60 degrees about 45 degrees. Viewing distances increased from about 0.5 mm to about twice the distance in vivo in an animal as the FOV decreased from 80 degrees to 45 degrees. While this increased the viewing distance through blood, it had too small of an FOV to be practically useful and was abandoned.

Later Experiments

Systematic experiments with lower FOV/NA were conducted in an in vitro blood fixture, where accurate measurements of distance could be made. The fixture used standard blood oxygenation techniques with flowing human blood passing through a chamber with an optical target and a port directly opposite it for insertion of the endoscope. The FOV was systematically reduced from 60 degrees and to 30 degrees. This effectively changed the acceptance angle, which is half of he FOV, from 30 degrees to 15 degrees. Viewing distances increased from about 0.5 mm to about 3 mm in the vitro blood fixture. This is a meaningful distance in coronary arteries since their sub-5 mm size would permit the entire circumference to be imaged.

### First Embodiment

The first embodiment (FIG. 3) is a light scanning probe inserted over the wire residing in a section of interest of a coronary artery. The section of interest would generally be a region of stenosis discovered in angiography. It could also be the region occupied by a stent to evaluate stent patency and early signs of stent restenosis. If vulnerable plaque burden estimation was the goal, the first centimeter of the main coronary arteries or sections of the carotid artery would be possible locations.

The probe is composed of an outer sheath containing two flexible fiberoptic fibers, one hollow wound spring for rotation and translation of the optical assembly, and a guidewire channel permitting it to passed over an indwelling guidewire. The first embodiment consists of only two fibers: an illuminating (4) and receiving (5) fiber. The receiving fiber (5) is connected to collection optics (7) and routed to an infrared detector (12) rather than an infrared camera. The probe (3) is inserted over a guidewire (15), which resides in a coronary artery section (1) with a 7 mm diameter. The catheter illumination is a polychromatic laser diode (6) emitting infrared light (10) with wavelengths from 800-1850 nm down a single fiber (4) with it exits the fiber (9). In this embodiment, the N/A is chosen to have an effective FOV of about 1 degree. The light (10) from the fiber contacts a rotating mirror (2) situated about 45 degrees from the catheter axis. The light (10) is thereby directed normal to the vascular wall. The backscattered light (28) is reflected by the mirror/prism (2) at position (29) where it is directed into a single receiving fiber of low-NA. The light travels down the receiving fiber (5) to an infrared sensor (12) and the intensity is recorded in a computer (13). This value is represented as a pixel on the infrared image (14).

The rotating mirror assembly is actuated and connected to the distal end of a spring in the catheter distal end. The typical speeds are 20-120 Hz. To capture the entire vessel wall without gaps, 120-1012 images need to be processed each revolution of the mirror assembly. 1 degree FOV and 360 images may not provide sufficient over scanned/sampled tissue area to provide high resolution image components, higher sampling such as 1012 samples may be required for highest quality

In one rotation of the mirror, a ring of tissue is imaged with the width (W) of the ring equal to (L)tan(0.5×FOV), where L is the distance from tissue. The mirror/optical assembly is then translated back using a worm-gear apparatus (20) to a position W cm from the previous image. The worm-gear apparatus (20) is located inside the catheter handle and is connected to the optical assembly with a wire. For a 7 mm diameter coronary artery, the 1 mm diameter catheter is typically about 3 mm from vascular wall. Each image ring width is about 0.03 mm. To image a 1 cm section would require about 300 rings. If averaging of the collected signals is used to eliminate sensor, mechanical, and optical noise. Using 10 revolutions to produce data for one revolution will require at least 3000 revolutions will be needed to image 1 cm of vessel. Depending on the speed of the wormgear, the entire section could be acquired in as little as 0.01×300 or about ⅓ to 3 seconds. The speed of acquisition thus permits multiple revolutions and use of signal averaging algorithms to increase the signal strength. If longer sections greater than 1-2 cm require examination, the physician can manually retract the catheter guided by centimeter markers or stops. The handle could also incorporate an automatic retraction of a fixed amount (say 1 cm), controlled by a button on the handle or a touch-sensitive icon on the image display.

This rotating nature of the embodiment permits signal averaging and spectrometric analysis. Since the mirror rotates around 60-120 hz, around 60 to 120 full-360-degree images are recorded each second. The main purpose of imaging a coronary artery is too accurately depict the vascular wall pre and post-procedure, In the pre-procedure examination, the vessel wall is imaged to discern the nature of the plaque and its position within the artery. A stent is deployed or an atherectomy perfumed and the arterial section is again imaged post-procedure to judge procedure effectiveness. In the case of stent deployment the stent apposition to vascular wall will be examined. If a portion of the stent is not apposing vascular wall, a condition called in-stent stenosis may be created in which the stent becomes plugged from reaction to the stent. This is particularly true for drug-eluting stents, which rely on endoscope face apposition to elute the drug deposited on the outside surface to the tissue. In the case of atherectomy devices, the post-procedure image will judge the completeness and possible complications of the atherectomy procedure.

The principles of normal light application received with a low-acceptance receiver can also be achieved without the use of any optical waveguides whatsoever. Endoscopes have been constructed with both area arrays (Adler) and the illumination source at the distal end of the catheter. The detected values are then transmitted by electrical wires to the computer. This is of particular importance in this invention, because detection can be achieved with a single optical detector, which can be highly miniaturized.

The small size of the probe (3), containing only two fibers and wire rotating the mirror (2) can be constructed to be very small in diameter. Such a small-diameter device (˜3 F or 9 mm) could easily be incorporated into the atherectomy device and positioned to image the active tissue removal part of the device as it shaves plaque in real-time. Even in this application, multiple passes could be accomplished while still giving the appearance of real-time operation.

Returning to the probe (3) illuminating a section of the internal wall of a coronary artery (1), if a higher quality image is desired or if greater penetration depth is desired, the data can be accumulated over more than one rotation. For example, in one second, a mirror spinning at 120 hz could accumulate 120, 360-degree images of a ring in the coronary artery section (1).

With the mirror/prism at an angle of 45 degrees with respect to the probe axis, light will be directed approximately 90 degrees with respect to the vessel wall. If the mirror/prism were set at a smaller angle, such as 30 degrees, the incident light would strike the vessel surface ahead of the optical head at a oblique angle of 115 degrees. In FIG. 2, two light probes (44) are shown near an arterial bifurcation. The lower probe has the mirror (2) set at 45 degrees, while the upper probe has the mirror (2) set at 60 degrees. In an artery 6 mm in diameter and a 1 mm diameter light probe 1-4 mm from arterial wall of the artery, the surface of the artery would be between 12 mm away from the light probe. If the mirror were at 30 degrees, relative to the probe axis, the circumferential ring of tissue would be ahead of the center of the mirror by minimum amount of 2 mm×tan(15 deg)=2×0.268=0.52 mm to 4 mm tan(15)=1.04 mm ahead of the mirror/prism center. If the FOV were 30 degrees or an acceptance angle of 15 degrees, then the width of the imaged ring would be 1.04 mm for the tissue 2 mm away from the probe to 2.08 mm for the tissue 4 mm away from the probe. Thus, the edges of the image field would span about 1-2 mm. Adding to that 0.5-1 mm offset in image center produced by the 30-degrees in front of the distal end of the probe, an object could be observed 1.5-3 mm from the light probe with a mirror oriented at 30 degrees relative to the probe axis. Tissue, such as that between ostia of vessel bifurcations would be more prominent than vessel wall since the light would be reflected closer to 90 degrees. The resultant concatenated ring image would show a brightly lit bifurcation septum with holes on either side. This canted-mirror embodiment permits navigation of the vascular tree, imaging the bifurcations ahead of the probe distal end. A curved guidewire can be advanced and also be viewed in the canted-mirror ring image as a luminous line, due to the reflective nature of metal.

A physician could navigate the vasculature using a mirror positioned at 30 degrees relative to the probe axis. As a bifurcation was observed on the light scanner image monitor a few millimeters ahead of the probe distal end, the guidewire is withdrawn into the probe and probe is manipulated, guided by the light scanner image, be in front of the desired branch. The guidewire is extended into the desired branch and the light scanner advanced over the guidewire into the desired branch artery.

In this embodiment, the translation means was accomplished by a worm gear apparatus. Other means of “effective” translation can also be achieved by:
1. Manually withdrawing the probe by the physician at the probe handle and sensed by a position sensor in the probe handle.
2. Automatically altering the angle of the rotating mirror. For example, the first mirror revolution is at a 45 degree angle relative to the probe axis. The second revolution would be at 47 degrees, illuminating a ring of tissue ahead of the tissue illuminated in the first revolution. A ring more proximal to the first ring is achieved setting the mirror angle at 43 degrees. This is similar to direct translation and would be limited by less resolution as the mirror angle deviates from 45 degrees.

A spectrometric derived chemical analysis of the coronary artery section could also be obtained by placing a diffractive element on the proximal end of the receiving fiber (5). A diffractive element in close contact with the proximal end of the receiving fiber (5) separates the received signal into many signals, each centered at different wavelengths. The diffractive element divides the incoming light into wavelength regions. These signals are sent to a linear array or area array (18) with each element of the array corresponding to reflected light in different wavelength regions. Even though the intensity of each of the divided signals is fraction of the total signal, averaging techniques improves the signal count of each divided signal.

The block diagram of the system is shown in FIG. 4. Starting at the vessel wall (1) and ending at the display (14), the block diagram shows the bi-directional hardware interfaces. The controls will all be issued from the CPU/User Interface (13) and a detail software explanation is not given in this description since is not a subject of the patent claims. However some software functions will be named.

The vessel wall (1) has several layers of bio-material that give unique energy reflections. The reflections can be thought of as signatures that can be broken down into components of texture (generally the physical profile of hills and valleys of each 360 degree scan) and spectral variations due to absorption due to the bio-components. The tissue has flowing blood present with no artificial diluents. The general reflective signal contains the scattering and absorption effects of a double pass through the flowing blood and the signature of the tissue layers.

Photons (10) leave the optical head (62) and reflect from the vessel wall and are received (61). The optical head (62) or the distal catheter tip produces a very narrow lateral angular photon beam width and is directed azimuthally in a 360 degree scan. A series of these scans at different axial locations describe the amplitude map of a section of vessel. The energy from the distal optical head (62) is transferred by optical fibers (4, 5) or waveguides. There are other descriptions in this patent that do not require optical fibers as the energy transfer method. The photons have two paths in the system description. Both are shown on opposite sides of the block diagram.

The probe handle (63) and distal shaft contain the rotational actuator shaft for the reflective optical component producing the 360 degree scans, and the transmitting and receiving optical fibers. The mechanical design is such that no mechanical interference is produced by the rotary motion of the shaft. The optical fibers are typically low NA energy guides. The axial motion mechanism is contained in the proximal portion of the catheter handle (63). The catheter handle (63) will have control surfaces for the mechanical and some of the optical functions. The proximal catheter shaft will have connections to the system module. The system module will typically contain the Energy Module (67), Energy Controller (68), Data Collection (69), and rotational motion control (66).

The system module will be connected to a User Interface controller. The User Interface Controller contains an active display (14) and CPU (13) to process the incoming signals and generate commands for the system module per the User selections.

The Energy Module (67) is the optical/mechanical interface between the optical fibers and the Energy Controller (68). On the transmit side of the module the optical fiber (4) is mechanically connected with specifically designed connector for precision axial and lateral positioning. The accurate positioning is essential for maximum energy transfer between optical lenses and the fiber tip. The optical lens design relays the high NA input energy efficiently to the plane of low NA fiber tip. On the receiver side of the Energy Module (67) a similar transfer takes place. The receiver optical fiber (5) is connected to the module with a similar connector for mechanical accuracy and the optical relay transfers the energy to a suitable distance to a mechanical interface.

The Energy Controller (68) on the transmit side has a laser/light source module positioned accurately with respect to the input interface of the Energy Module (67). The laser/light source module is self contained units that can be replace by other energy modules as the User desires and the design of the mechanical interfaces preserves the necessary alignment. The laser/light source modules can contain single or multiple emitters and collection optics necessary to match the NA needed by the Energy Module (67). The receiver side of the Energy Controller (68) has an optical relay that accepts the output of the Energy Module (67) and transfers at the proper magnification for detector module. The detector module is a replaceable module as indicated by the User. The detector module can have a single pixel sensor, linear array, 2D array, or custom sensor to receive the transferred energy. The optical interface of this side of the controller can contain dispersive and/or polarizing spectral components to alter the raw signal from the Energy Module (67). The Energy Module (67) is electrically connected to the Data Collection (69).

The Data Collection (69) on the transmit side issues the proper control signals to the laser/light source module. These signals could contain frequency, duty cycle, peak current, and average current commands to match the User's desired energy profile. The receiver side can perform different tasks depending on the User's desired configuration. For a single detector pixel in the Energy Controller (68) the detector bias and temperature control has to be maintained to ensure the desired amplitude response. A BIT (built-in test) may be present depending on the long term stability of the sensor. The signals will be digitally converted as necessary to at least 16 bits and potentially processed in an S/H (sample and hold) device for averaging and formatting. Some of the detector modules may contain digital converters; in this case the S/H stage will be bypassed. Common to both sides of the Data Collection (69) will be the reformatting of signals to include scanning information such as angular position, scan number, axial position, wavelength, and energy input. This formatting information will be used by the CPU/image Processing unit (13).

The CPU/image Processing (13) will be a commercially available PC or laptop computer. There will be several resident programs operating to maintain control of the catheter system. A system control will translate the User commands to all module control programs and maintain good operation conditions. In the event of any module malfunction detected by monitoring programs an energy shutdown will occur and the User notified for corrective action. The energy generation will not function until all malfunctions are corrected. The system control can be User/Technician examined to display state conditions and run analysis routines. Image processing control is a User selectable option. The image processing program allows the User to select a single processing routine or a series of processing routines. An advanced User option is available for experienced Users to modify specific processing parameters in the desired processing routines. Some of these parameters may include signal averaging definition, wavelength or wavelength bands selection, laser/light source energy levels or time profiles, speed selection of the 360 degree rotation and axial motion controls. These User generated routines can be saved for later uses. All the data (raw and processed) will be saved in time dated files for post analysis. The outputs of the image processing will a constructed image or images selected by the User. An optional analytical data processing can be User selected for display with the real time images. Such optional processing could include energy/wavelength in current use, various comparisons to stored information related to normal tissue parameters, and relative axial or radial positions. The displayed images will be stored in a file for post analysis and allow Users to review the exact displayed image. The display or computer screen may have a touch sensitive control functions for easier User interface.

In general use it is expected the physical system design will be modular or ease of storage when not in use. The modular design can facilitate the substitution or changing User selected modules. The CPU/image Processor (13) can be used off line as a research tool for reviewing stored files from procedures or bench testing. All system modules will have suitable interfaces for testing by technicians. The catheter design will include connectors that are relatively easy to install by gloved technicians and Users. The design of the catheter shafts are disposable and the handle with controls will be reusable and sterilizable for a limited number of uses. The tractability of the control surfaces will be accessible through a thin sterile polymer shield with a gloved User. The reuse of catheter shafts is dependent on manufacturing and subject regulations.

In general operational conditions the Simplex catheter is a polychromatic energy delivery device. The term polychromatic is used because a continuous light source with a narrow bandwidth filter can be used to produce near monochromatic conditions. For monochromatic light sources with high energy production lasers/laser diodes are suggested. Typical ‘white’ light sources nominal narrow bandpass energies are much lower amplitudes when compared to laser devices. Some distances through blood may require the use of lasers devices for highest amplitude return signals. It maybe necessary for some catheter designs to incorporate larger diameter transmit fibers to capture sufficient energy for the vascular scans depending on anticipated distances through blood. These transmit fibers may have some influence on the mechanical OD of the catheter shafts if the OD's exceed 25 microns. Future uses (undefined at present) of energy transmission through blood may require the employment of single mode fibers to preserve the highest possible energy amplitude of polarized energy prior to entering the blood stream. In this case the OD of the catheter shafts will be influenced by the fiber size.

In the Simplex operation the speed of rotation will be User selectable to allow the possibility of maximum energy transfer to each radial position in the 360 degree scan. Slowing the scan rate will result slower updating to the information displayed by the CPU, so the User should be aware of this limitation when using maximum energy per scan. The scan mechanism will only rotation in one direction or remain fix in one position. There are no other states of operations. This choice is made because of the mechanical inaccuracies of reversing the rotational mechanism for less than 360 degree scans. If partial angular scans of vessel tissue are desired then a manual scan is suggested. If a defined angular (less than 360 degrees) scan can be defined, then the energy will only be active during that scan position of each revolution. This can be used for a continuous, reliable scan of a particular section of vessel wall.

If a polychromatic laser is selected for the energy source the User will need to observe the images and analysis if sufficient energy at each wavelength is being processed. Polychromatic laser have high energy amplitudes at very short durations. It may be necessary to increase the number of 360 degree revolution per axial location and increase the integration time per pixel at the detector to capture sufficient energy for analysis. Another option for higher energy levels for polychromatic analysis, if known wavelengths (say up to five) can provide sufficient analysis then discreet emitters can be used. This approach to polychromatic light sources has been practiced for many years by laser diode manufactures. Typically arrays of emitters are combined in one emitter stack either in one dimension or two-dimensional positioning to provide several high energy wavelengths from one small area. Maximum efficiency is that all the emitters are operated with each pulse. The smaller the emitting area the easier it is to collect the energy efficiently. Designing arrays for the insulation material will thermally limit individual wavelength selection, and create inefficiencies in the driver current. Collecting the emitted energy from these arrays has several optical solutions.

If a polychromatic source is used, the reflected light can be separated into wavelength bands using a optically dispersive element. This permits chemical analysis by collecting images in wavelength regions with high absorptions for the chemical/biological entity of interest. For example, lipids have absorpbance peaks around 1700-1800 nm. Images in this region will be more sensitive to lipids.

FIG. 5 is a schematic of the technique of hyper-spectral imaging. Proximal to the collection fiber after connecting to the Energy module (67) will be an optical dispersive element, such as a grating to separate the polychromatic energy into wavelength bands. The optical collection lenses after the dispersive element will collect the energy and image the bands onto a linear/area area for collection. The remainder of the modules will prepare the signal for processing by the CPU. The image processing program for the spectral analysis (hyper spectral imaging) will sort each wavelength and amplitude per pixel per 360 degree per axial location. Knowing the distance through blood and typical reflective values for normal tissue for this patient allows the program to calculate a relative absorption for each wavelength from the tissue. Depending on the chemical analysis needed to be preformed on the tissue these absorption values can be used to determine unique spectral signatures after the affects of blood has been eliminated from the amplitudes. The spectral amplitudes are stored in the CPU with the angular, axial, and radial position data, so a three dimensional representation of the vessel section can be displayed. The areas in the image that have particular chemical signatures can be colored different from the nominal vessel tissue.

In the Simplex catheter design when polychromatic energy is used for chemical analysis and hyper-spectral imaging the optical head design will include a small ultrasonic transducer for radial distance determination for each value in the 360 degree scans. This distance information will be stored with the amplitude and position data at the Data Collection module (69) and transferred to the CPU/Image Processor (13). Any spectral data gathered without the radial distance information can only be used for relative amplitude comparisons with each wavelength, because the tissue absorption cannot be calculated without distance. The special case of zero distance where the optical tip is in contact with the tissue can the absorption be calculated.

### Second Embodiment

While the first embodiment images the entire vessel wall over a 1-2 cm arterial section, the dimensions of the artery are unknown. Dimensions are required to present accurate three-dimensional images of the arterial section. Also, arterial dimensions are important in choosing the proper stent size.

Referring to FIG. 3, sizing is easily accomplished by placing a piezoelectric transducer in close proximity to the optical assembly. For example, it could be placed adjacent the optical fibers, slo pointing to the mirror/prism. At the same time an infrared light beam is directed at an arterial segment by the rotating mirror, the mirror also reflects the ultrasound signal produced by the piezoelectric transducer.

Conversely, an ultrasound transducer could direct the ultrasound 180 degrees from the infrared light (FIG. 6). As shown in FIG. 6, the piezoelectric transducer (32) is mounted on an outside face of the mirror/prism where it directs ultrasound (94) in an opposite direction from the light (10). The received ultrasound signal ultrasound is detected by the piezoelectric transducer and an electronic signal generated. This signal is transmitted to an ultrasound data acquisition system (91), whereby the ultrasound reflections are recorded in all positions as the mirror/prism rotates. The distance vector calculations will need to be coordinated with the optical signals at the correct angular heading. Once these signals are coordinated a combination IVUS-light image is obtained of the vessel. This permits vessel cross-sectional views from the IVUS portion together with high-resolution surface images produced by the light scanner. The IVUS and light image could be combined. For example, the IVUS image could be in greay scale, while the light image could be colored red. Or the images could be side-to-side as well. The combination is attractive because it permits cross-sectional views combined with high-resolution surface detail. In addition, incorporating ultrasound permits accurate distance measurements of the distance between the probe and biological surfaces. This then permits the creation of 3D construction of the blood vessel, where it can be rotated, split open or other tomographic views.

A portion of the second embodiment could utilize ultrasound primary reflection only to measure distances and produce the traditional ultrasound views of vessels. If the user sees relevance the two views, optical and ultrasound, both can be displayed separately.

A more sophisticated approach would be to overlay the entire image produced by ultrasound onto the infrared image. This could be presented as a tomographic image overlaid with surface optical amplitudes characteristic of the chemical composition.

The display of the image for the user will have image processing tools available to show the 3D image of a vessel and various cross sectional views in suitable orientations. The 3D image can be unfolded from a circular to a flat 2D image. If only 2D angular sections of the vessel are of interest then the user can specify only those 2D images of interest for display.

### Third Embodiment

A multi-fiber approach demonstrates how 10 scan lines can be produced instead of one pixel spot. 10 lines was chosen as an arbitrary number for demonstration only any practical number of lines can be designed. The number of lines are limited by the fiber bundle diameter and individual fiber OD. In FIG. 7, a multi-fiber probe (92) has multiple emitting and receiving fibers (93). Ten lines of light (10) at 36 degrees apart are shown. Each line is produced by the projection of 10 receiver fibers that are adjacently located in the fiber bundle. The reflective surface of the mirror is a complex facet optical design to keep the line projection straight while the mirror is rotated. With 10 facets on the mirror each 36 degree angular section of a vessel is scanned 10 times per revolution.

With 10 scanned images of each angular section an averaging of the optical data can be accomplished with one revolution instead 10 revolutions with a single pixel detector.

The ultrasound distance measuring data will be to each line rather than each pixel. Since the line length is small due to the distance to the wall of the vessel the distance will represent an average distance to the line segment. This will be considered a minor disadvantage compared to the single pixel approach where the distance to each pixel project is known. However, if the ultrasound transducer's phase can be translated along the line then accurate distance measurements for each fiber projection can be obtained.

The images of the scanned lines can be captured on a linear array or an area array detector. Most IR area array detectors have ROI (region of interest) functions which will confine the data collection to the line image and not the surrounding pixels. In the case where a spectrally dispersive element is used with the line images then the IR area array is well suited to collect all the spectral images of scanned lines.

### Fourth Embodiment

The use of area (2D image sensors) arrays is common in today's endoscopes. The majority of today's endoscope are used in the visible light wavelengths, but changing the sensor to an IR area array and the illumination to a suitable IR energy source will allow some endoscopes to view tissue at IR wavelengths. To ensure best IR results fiber endoscopes should use image fiber bundles where the individual fiber OD's are equal to or larger than 5 microns. This ensures the efficient passage of IR energy.

In general, visible and IR wavelengths, large FOV endoscopes cannot view successfully through blood due to light scattering properties of the red blood cells. It has been explained in this patent's text how to reduce the collection of diffuse photons by using optics or fibers that only accept low NA light energy. When this principle is applied to larger FOV it is necessary to use telecentric optical collectors for FOV's larger than a few degrees. Telecentric as applied to optical imagers or collectors implies the system's aperture stop has been adjusted to only allow the principle rays from the object of interest to enter the imaging fiber bundle parallel to the optical axis. If an area array is in place of the fiber imaging bundle then the principle rays enter it normal to its plane of pixels. This application of the telecentric principle allows a finite FOV to be collected.

To ensure that the diffuse photons are rejected from the FOV collected by the area array or imaging fiber bundle the NA surrounding each principle ray has to be controlled or limited to some small value. In the case of imaging fiber bundles the NA of the manufactured fibers is selected to be small by the choice of the indices of refraction of the core and clad materials. In the application of an area array the size of the ID of the aperture stop is chosen to have a value when combined with the EFL of the collection optics yields a large F/#.

A specific example is using a fiber imaging system in a vessel such as a coronary artery. The distal collection optics uses a rotating mirror to scan the FOV in a 360 degree path collecting reflected energy from the vessel walls. As a comparison to the Simplex Model described in the patent where the collection fiber face is projected onto the vessel wall, the fiber imaging bundle will project many fiber faces onto the vessel wall. The total projected area of fibers is large compared to a single fiber so more of the vessel wall is imaged. It sufficient to collect fewer image samples as the mirror rotates through 360 degrees making the image processing task less complicated than the Simplex Model or the Scanned Line models. If 4-10 image frames are collected with each 360 degree revolution, then several revolutions will be sufficient to accomplish an averaging processing routine to reduce noise. These averaged frames can be processed by most frame grabbers at rates of 30-1000 frame per second.

Using a rotational rate of 120 Hz and 5 rotations to average the collected frames for each 360 degree strip of vessel wall images give an effective axial scanning rate of 22 Hz. For calculation purposes assume the fiber projected spot is 0.30 mm. Using 22 Hz×0.30 mm=6.6 mm/second of vessel wall scanned. Again using 10 averaged frames per revolution×22 Hz=220 frames per second, a value that is well within the capabilities of frame grabbers to process. This application of a scanning catheter will allow a user to view an extensive section of vessel wall every few seconds. If dispersive or diffractive spectral elements are used in the optical path then a higher rate frame grabber must be employed to process a hyperspectral imaging chemical analysis to test for pathologies in the vessel wall.

### Fifth Embodiment

Besides assessing the pathological condition of a vascular surface, the scanning imager with the dispersive element can also be used for cancer detection in other tubular-like structures. These structures include, but are not limited to the esophagus, colon, stomach, fallopian tubes, lung, trachea etc. The light scanner probe is inserted internally to the internal organ. For example for the stomach, it is placed in the mouth and routed to view the stomach, the lungs accessed from the mouth. While the stomach is not tubular, it is an example of an elongate organ. Because light is directed perpendicular to the probe axis, most of the wall of the stomach will have light directed roughly normal (+/−30 degrees) to the surface. Accumulating light amplitudes with multiple rotations permits signal accumulation and averaging techniques which enhances faint signals.

Besides the conventional entry sites for endoscopes, the scanning imager can access internal organs through the blood supply, because of its ability to image through blood. For example, to access the liver, a guidewire is inserted into a vein in the groin and with fluoroscopic guidance is routed to the inferior vena cava (IVC). The portal vein leading to the liver is a large orifice on the side of the IVC. It can be imaged by puffing radio-opaque dye and maneuvering the guidewire into the portal vein ostium. The guidewire is advanced into the liver. The scanning imager is inserted over the guidewire, where it is routed to the liver. Once inside the liver, the guidewire can be routed to various vein branches. With this technique, various sections of the liver could be explored, both with the simplex probe embodiment and the spectrophotometric embodiment. Other organs reached from the IVC include the pancreas, kidneys and spleen.

Infrared light is useful in cancer detection because infrared light penetrates tissue more deeply than visible light allowing cancerous cells within the tubular structure to be detected. The infrared spectrum from 1300-3600 nm contains absorbance maxima for many biological components. Cancer cells are of a different chemical composition than normal cells. The presence of cancerous cells can be imaged by overlaying on the image obtained from polychromatic light, an image illuminated by a wavelength at an absorbance peak corresponding to a prominent chemical entity of the cancerous cells. This can be accomplished using a band-passed polychromatic source or a monochromatic laser or LED. Even structures without fluids could be examined in the infrared using the diffractive element and the methods above. In the wavelength regions where cancer absorbance peaks are present, the scanning device of this patent application could have an algorithm to emphasize the cancer-absorbance-peak wavelength by subtracting out the background produced by other wavelengths.

Also important in cancer detection are the detection of fluorescent dyes, such as indocyanine green, which preferentially attach to cancerous cells. Visible spectrum dyes are also available. Hexvix, or hexyl aminolevulinate, is similar to a chemical found naturally in the body and contains porphyrins. Cancer cells absorb this substance faster than healthy cells, and they turn fluorescent pink when the cystoscope light changes from white to blue.

The dye is administered to the patient or directly in the organ and when directly illuminated at a certain wavelength (760 nm for indocyanine green) it emits light at a higher wavelength (810 nm for indocyanine green). The scanning imager with indocyanine green infusion could also detect this fluorescence by emitting a monochromatic or narrow wavelength region centered at 760 nm and the received wavelength region could be made narrow and centered on the emitted fluorescent wavelength of the dye consumed by the patient, which is preferentially absorbed by cancerous tissue.

The advantages in the light scanning imager over an endoscope are as follows: (1) the entire elongate organ structure is evenly evaluated rather then just the portion within the FOV. This permits the underlying cancer cell pattern to be elucidated. Recently, it has been reported that colonoscopy's suffer from a rightsided “blindness” since the anatomy of the right side of the colon prevents the endoscope from adequately recognizing polyps on the right side. The study suggested the reasons for this included “first, some colonoscopies considered “complete” may not evaluate the entire right colon. Second, bowel preparation may be worse in the right colon. Third, right and left colonic cancers and polyps may differ biologically. Right-sided growths may be less likely to have a fleshy stalk and are occasionally flat, which makes them harder to identify and remove, or they may grow more rapidly”. The scanner images the entire circumferential ring. Translating the probe permits the entire colon to imaged with similar light conditions and mostly normal to the colon surface. (2) Because of the small FOV, the infrared scanning imager can create a higher light flux at the particular piece of tissue imaged, since only a small section of tissue is illuminated at a particular time. FIG. 9A illustrates the order of magnitude difference in emission angle between a conventional endoscope (47) and a scanner (44). The spot illuminated spot size for the scanner (78) is much more concentrated than the more diffusive endoscope spot size (77) (3) the light is directed in a near-normal direction to the elongate organ surface. More intense infrared light applied near-normal to the tubular surface would better detect cancer cells on the outside of the tubular structure. It is also advantageous in exciting fluorescent dyes residing in cancer cells not on the tubular surface. (4) faint signals can be accumulated by making multiple rotations to create a visible image on the computer monitor. This condition is shown in FIG. 8 A, which depicts a traditional endoscope (47) and a light scanning probe (44) inside an elongate internal organ (70). There are small cancerous lesions on the inside wall (76), inside the wall (74) and on the outside of the wall (76). The endoscope has an FOV (71) of about 60 degrees resulting in diffuse light emission. The light scanning probe (44) has a narrow FOV (72) resulting in a much higher light flux at the surface of the organ. While the endoscope might detect lesion on the inside wall (76), light strikes the lesion at a grazing angle, which limits the returned reflective light. Lesions on the inside or the outside surface would likely not be detected because of lack of normality and low light flux at the surface.

The light scanning probe (44) is much more likely to detect the lesions inside the wall (74) and on the outside of the wall (76) because the light flux at the surface is much greater for the light scanning probe (FIG. 8 B, 77) than for the endoscope (FIG. 8 B, 78). In addition, as shown in FIG. 8C, whereas the light scanning probe projects light about perpendicular (80) to the surface (70), the endoscope is at an oblique angle (79). If infrared light is used as well, the surface (70) will be penetrated more greatly by the light. For example light around 800 nm is highly penetrable of tissue. Enhanced light detection results in improved image contrast. Moreover, the scanning imager images the entire surface of the tubular structure over several centimeters, whereupon it can be advanced or retracted to view the next centimeters. Thus, an automatic scan is made of all the structure surface and presented as a “cancer map”. This principle has application in the visible and the infrared light regions.

In contrast to an endoscope, the light scanning probe would apply more light to each tissue segment and would image the entire tubular structure over 360 deg over several centimeters. On the other hand, the endoscope would have to be directed by the physician to the cancer cell site. Moreover, the random light incidence created by a hand-manipulated endoscope would not evenly illuminate all sections of the tubular structure. Cancerous areas could be missed due to the uneven illumination.

The disclosures teach a light scanning system whereby light is directed to at a rotating mirror, strikes the vessel/elongate organ about perpendicular to the surface and received by low-angle-of-acceptance receiver such as an optical fiber with low-NA. There are another of configurations that can be constructed using these same principles.
(1) The receiving fiber can be replaced by a single detector, linear array detector or area array detector on the distal end of the probe. The detector could be mounted perpendicular to the probe axis, not requiring received light reflection from the mirror/prism or it could be in parallel to the probe axis in close proximity to the projecting fiber, redeiving the reflected light from the mirror/prism. The detector uses electrical wires to transmit the detector data through the probe handle to the console.
(2) The emitting fiber can be replaced by a light emitting diode or monochromatic laser diode or polychromatic laser diode. The light source could be mounted perpendicular to the probe axis, not requiring light reflection from the mirror/prism or it could be in parallel to the probe axis in close proximity to the projecting fiber, projecting the reflected light to the mirror/prism, where it is reflected approximately normal to the probe axis. Electrical wires from the light source route out of the proximal end of the probe to the computer console.
(3) Fiber-less system using both a detector and a light source at the distal end of the probe. The detector uses electrical wires to transmit the detector data through the probe handle to the computer.

The combination of perpendicularity and a low-angle-of-acceptance receiver permits vascular wall to be imaged through flowing blood. Spectral analysis of tissue with the dispersive element is possible for the examination of cancer or atherosclerotic plaque. The advantages of a scanner over a conventional endoscope for the examination of a vascular wall or elongate internal organ include the following:

| ENDOSCOPE PROBLEM | SCANNER ADVANTAGE |
|---|---|
| Non-systematic Examination | Scanner views entire circumferential surface over the translation |
| | distance, providing an image of the entire wall-nothing missed. |
| | Permits historical comparisons with previous measurements |
| Limited FOV | Effective FOV is 360 degrees |
| Forward Viewing | Scanner views 90 degrees from the forward direction in a direction |
| | approximately normal to the vessel or elongate wall |
| Blinded by Scattering Media | Scanner views through blood because of normality to surface and |
| | low-FOV receiving waveguide |
| Unable to Make | Scanner uses dispersive element to create wavelength bands, |
| Spectrophotometric Measurements | which are transmitted to a linear array camera. Wavelength |
| | regions of interest can be highlighted on the broad wavelength |
| | band image. |
| Cannot Measure Distances or | When an ultrasonic transducer is also incorporated, the actual |
| Determine Object Size | distance can be easily measured by locating the primary reflection |
| Inability to employ Signal | Scanner can accumulate multiple images of the same scene with |
| Averaging Techniques | multiple rotations prior to translation. |
| Inability to view Fluorescent Dyes | The normal light application and small light emission angle permit |
| | very high light fluxes permitting greater depth in activating |
| | fluorescent molecules. Greater sensitivity is achieved when |
| | normal to the surface. Additional sensitivity is achieved by |
| | accumulating light intensity over multiple rotations |
| Inability to Construct 3D | With the addition of an ultrasound transmitter/receiver, distances |
| Tomographic Images | can be determined from the principle refection, permitting the |
| | construction of 3D tomographic images |

We claim:

1. A method of imaging a circumferential ring of the vascular wall of a blood vessel, through flowing blood, comprising:
inserting a flexible light probe into the vessel
projecting light onto a mirror/prism which directs the light approximately normal to the vessel surface, such that some light is reflected, minimally scattered or multiply scattered from the vessel wall
receiving the reflected and minimally scattered light preferentially from multiply scattered light by receiving the light with a low-acceptance angle criteria
transmitting the received light to a light detector
recording the light amplitude in computer memory
rotating the mirror/prism about the probe axis to successively record a ring of light amplitudes around the entire vessel circumference
concatenating the recorded light amplitudes in all rotational positions with a computer
whereby, an image of a circumferential ring of vessel wall is obtained.

2. The method of claim 1 wherein the projecting light is in the infrared region 800-3600 nm.

3. The method of claim 1 further comprising a polarizer element placed over the emitting and receiving light beams.

4. The method of claim 1 wherein the low-angle light acceptance angle is less than 15 degrees.

5. The method of claim 1 further comprising a translation element to image and concatenate multiple rings and provide a composite image over the translation length.

6. The method of claim 1 further comprising the addition of an ultrasound transducer to measure the distance to the vascular wall and thereby create three-dimensional images

7. An intravascular probe comprising:
at least one illuminating optical waveguide connected to a light source and terminating in front of a rotating mirror/prism, whereupon the light is directed approximately normal to the vascular surface
at least one receiving optical waveguide with low numerical aperture (NA), in close proximity to the illuminating waveguide, receives the backscattered light from the vascular surface, off the mirror/prism and transmits it to a light detector
computer memory to record the light amplitude and mirror position
an actuator to rotate the mirror/prism about the vessel axis to successively record a ring of light amplitudes around the entire vessel circumference
a computer to concatenate the recorded light amplitudes in all rotational positions
a display to present an image of the circumferential ring of vascular wall.

8. The intravascular probe of claim 7 wherein the projecting light is in the infrared region 800-3600 nm.

9. The intravascular probe of claim 7 further comprising a polarizer placed over the emitting and receiving waveguides.

10. The intravascular probe of claim 7 where the illuminating and receiving optical waveguides are optical fibers or hollow waveguides.

11. The intravascular probe of claim 7 further comprising a translator actuator to move the optical waveguides relative to the vascular wall to image multiple rings and concatenate them to provide a composite ring image over the translation distance.

12. The intravascular probe of claim 7 further comprising the addition of an ultrasound transducer to measure the distance to the vascular wall.

13. The intravascular probe of claim 7 where the low-NA receiving fiber(s) is less than 0.1.

14. A method of imaging the chemical/biological composition of a circumferential ring of vessel or internal elongate organ wall comprising:
inserting a light probe into the vessel or internal elongate organ
projecting light onto a mirror/prism which directs the light approximately normal to the vessel/elongate organ surface, such that some light is reflected, minimally scattered or multiply scattered from the vessel wall
receiving the reflected and minimally scattered light preferentially from multiply scattered light by receiving the light with a low-acceptance angle criteria
transmitting the received light to a dispersive element
focusing the light from the dispersive element to an area array camera
recording the light amplitudes in each wavelength region in computer memory
rotating the mirror/prism about the probe axis to successively record a ring of light amplitudes for each wavelength band around the entire vessel circumference
concatenating the recorded light amplitudes for the received infrared light in all rotational positions for each wavelength band
highlighting particular wavelength bands
whereby an image of the circumferential ring of the vessel/elongate organ is obtained highlighting the position of wavelength bands corresponding to the chemical/biological entity of interest.

15. The method of claim 14 wherein the low-angle light acceptance angle is less than 15 degrees.

16. The method of claim 14 further comprising a translation element to image and concatenate multiple rings over the translation distance.

17. An intra-vessel or intra-elongate organ probe comprising:
at least one illuminating optical waveguide connected to an infrared light source and terminating in front of a mirror/prism, whereupon the light is directed approximately normal to the vascular surface
at least one receiving optical waveguide with low NA in close proximity to the illuminating waveguide, which receives the backscattered light reflected by the mirror/prism from the vascular/organ surface, and transmits it to a light detector, such that some light is reflected, minimally scattered or multiply scattered from the vessel wall.
transmitting the light to a dispersive element which separates the light into wavelength bands
focusing the wavelength bands onto an array camera
a mirror/prism rotation actuator
rotating the mirror/prism about the vessel axis to successively record a ring of amplitudes for each wavelength band around the entire vessel/organ circumference
a computer to record each light amplitude measurement in all rotational positions
a display to present an image of the circumferential ring of vascular wall, highlighting wavelength bands corresponding to a chemical/biological entity of interest.

18. The method of claim 17 where multiple images are recorded of each tissue segment and the images are accumulated or averaged.

19. The intravascular probe of claim 17 where the illuminating and receiving optical waveguides are optical fibers.

20. The intravascular probe of claim 17 further comprising the addition of an ultrasound transducer to measure the distance of the vascular or organ wall

21. The intravascular probe of claim 17 where the low-NA receiving fiber(s) is less than 0.1.

22. A method of imaging the fluorescence emission of a circumferential ring of an internal elongate organ where some cells contain the fluorescence molecule comprising:
inserting a light probe into the internal elongate organ via a blood vessel or other passageway to the organ
projecting monochromatic light at the fluorescence-inducing wavelength at a low-emission angle onto a mirror/prism which directs the light approximately normal to the vessel/elongate organ surface,
receiving the fluoresced light with a low-acceptance angle criteria comparable to the emission angle
transmitting the received light to a light detector band-passed to accept only light with wavelengths near the fluorescence emission wavelength
recording the light amplitude(s)
rotating the reflective element about the vessel/elongate organ axis to successively record a ring of light amplitudes around the entire organ wall circumference
concatenating the recorded light amplitudes for all rotational positions
whereby an image is obtained of the fluorescence emission in a circumferential ring of tissue in the organ wall.

23. The method of claim 22 wherein the low-angle light acceptance angle is less than 15 degrees.

24. The method of claim 22 further comprising a translation element to image and concatenate multiple rings and provide a composite fluorescence image over the translation length.

25. The method of claim 22 where multiple images are recorded of each tissue segment and the images are accumulated or averaged
